(19) United States
(12) Patent Application Publication
(10) Pub. No.: US 2012/0150282 A1
(43) Pub. Date: Jun. 14, 2012

(54) IMPLANT HAVING A PACLITAXEL-RELEASING COATING

(75) Inventors: Nina Adden, Nuernberg (DE); Mark Steckel, Zuerich (CH)

(73) Assignee: BIOTRONIK AG, Buelach (CH)

(21) Appl. No.: 13/182,786

(22) Filed: Jul. 14, 2011

Related U.S. Application Data
(60) Provisional application No. 61/421,646, filed on Dec. 10, 2010.

(51) Int. Cl. A61F 2/82

(52) U.S. Cl. 623/1.38

(57) ABSTRACT

An implant having a coating or a cavity filling comprising a PLGA polymer and taxane embedded therein, the release rate of the taxane after day two after implantation being ≦400 ng/day for a period of more than 10 consecutive days, characterized in that the PLGA polymer has a ratio of monomer units to each other of 60-99% lactic acid units to 40-1% glycolic acid units.

### CROSS REFERENCE TO RELATED APPLICATIONS

This patent application claims the benefit of U.S. Provisional Patent Application No. 61/421,646, filed on Dec. 10, 2010, which is hereby incorporated by reference in its entirety.

### TECHNICAL FIELD

The present patent application relates to coated implants, for example to support vessels, hollow organs and vein systems (endovascular implants, such as stents), for fastening and the temporary fixation of tissue implants and tissue transplantations, but also for orthopedic purposes, such as nails, plates or screws.

### BACKGROUND

Implants are being employed in a wide variety of forms in modern medical technology. They are used, for example, to support vessels, hollow organs and vein systems (endovascular implants, such as stents), for fastening and the temporary fixation of tissue implants and tissue transplantations, but also for orthopedic purposes, such as nails, plates or screws. Frequently, only a temporary support or holding function is necessary or desired, until the healing process is complete or the tissue has been stabilized. In order to avoid complications resulting from the implants permanently remaining in the body, the implants must either by surgically removed again, or they are made of a material that is gradually decomposed in the body, this being referred to as biocorrodible. The number of biocorrodible materials that are based on polymers or alloys has increased steadily. Some of the materials that are known are biocorrodible metal alloys of the elements magnesium, iron, and tungsten. A particularly frequently used form of an implant is the stent.

The implantation of stents has become established as one of the most effective therapeutic measures for the treatment of vascular diseases. Stents have the purpose of performing a stabilizing function in hollow organs of a patient. For this purpose, stents featuring conventional designs have a filigree supporting structure comprising metal braces, which is initially present in compressed form for introduction into the body and is expanded at the site of the application. One of the main application areas of such stents is to permanently or temporarily dilate and hold open vascular constrictions, particularly constrictions (stenoses) of the coronary blood vessels. In addition, aneurysm stents are also known, which are used to support damaged vessel walls.

Stents have a peripheral wall with a sufficient load-bearing capacity in order to hold the constricted vessel open to the desired extent and a tubular base body through the blood continues to flow without impairment. The peripheral wall is generally formed by a lattice-like supporting structure, which allows the stent to be introduced in a compressed state, in which it has a small outside diameter, all the way to the stenosis of the particular vessel to be treated and to be expanded there, for example by way of a balloon catheter, so that the vessel has the desired, enlarged inside diameter. The process of positioning and expanding the stent during the procedure, and the subsequent position of the stent in the tissue after the procedure has been completed, must be monitored by the cardiologist. This can be done by imaging methods, such as X-ray examinations.

The stent has a base body made of an implant material. An implant material is a non-living material, which is used for applications in medicine and interacts with biological systems. A basic prerequisite for the use of a material as implant material, which is in contact with the surrounding body area when used as intended, is the body friendliness thereof (biocompatibility). Biocompatibility shall be understood as the ability of a material to evoke an appropriate tissue response in a specific application. This includes an adaptation of the chemical, physical, biological, and morphological surface properties of an implant to the recipient's tissue with the aim of a clinically desirable interaction. The biocompatibility of the implant material is also dependent on the temporal course of the response of the biosystem in which it is implanted. For example, irritations and inflammations occur in a relatively short time, which can lead to tissue changes. As a function of the properties of the implant material, biological systems thus react in different ways. According to the response of the biosystem, the implant materials can be divided into bioactive, bioinert and degradable/resorbable materials.

Implant materials for stents comprise polymers, metallic materials, and ceramic materials (as coatings, for example). Biocompatible metals and metal alloys for permanent implants comprise, for example, stainless steels (such as 316L), cobalt-based alloys (such as CoCrMo cast alloys, CoCrMo forge alloys, CoCrWNi forge alloys and CoCrNiMo forge alloys), technical pure titanium and titanium alloys (such as cp titanium, TiAl6V4 or TiAl6Nb7) and gold alloys. In the field of biocorrodible stents, the use of magnesium or technical pure iron as well as biocorrodible base alloys of the elements magnesium, iron, zinc, molybdenum, and tungsten are proposed.

A biological reaction to polymeric, ceramic or metallic implant materials depends on the concentration, exposure time, and manner in which they are administered. Frequently, the presence of an implant material leads to inflammatory reactions, the trigger of which can be mechanical stimuli, chemical substances, or metabolites.

A key problem of stenting into blood vessels is restenosis as a result of excessive neointimal growth, for example, which is caused by a strong proliferation of the surrounding arterial smooth muscle cells and/or a chronic inflammation reaction. Strategies to prevent neointimal growth focus on inhibiting the proliferation of surrounding cells through medication, such as by the treatment with cytostatic drugs. The active ingredients can be provided, for example, on the implant surface in the form of a coating. An active ingredient that is used frequently in this context is paclitaxel.

As an alternative or in addition, restenosis can be caused and/or promoted by the physiological effect of releasing the decomposition products of the stent, particularly the biocorrodible stent. For example, the decomposition of magnesium-containing stents creates an alkaline environment, which may result in increased muscular tension of the surrounding vascular muscle. As a result of such increased muscular tension, the cross-section of the stent may decrease or the stent integrity may even be lost prematurely. Drugs, which are known to reduce such muscular tension, are disclosed in DE 10 2006 038 235.

One of the problems is that the service life of degradable magnesium stents after implantation usually is, that they loose their integrity early and the vessel is not supported anymore. The vessel diameter reduces either because the vessel has not enough scaffolding ability after the injury it experienced upon implantation or because of the increased muscular tension described earlier.

It is the object of the present invention to reduce or prevent this early loss of vessel area of magnesium stents of the prior art.

### SUMMARY

This object is achieved by providing an implant having a coating or a cavity filling comprising a PLGA (polylactide-co-glycolide) polymer and taxane embedded therein, the release rate of the taxane after day two after implantation being ≦400 ng/day for a period of more than 10 consecutive days, characterized in that the PLGA polymer has a ratio of monomer units to each other of 60-99% lactic acid units to 40-1% glycolic acid units.

The present invention is based on the surprising realization that not every PLGA polymer typically used to coat implants is suited to release the taxane embedded therein at the desired long release rate needed to preserve the stent area during the magnesium degradation. It has been shown that only those PLGA polymers are suited in which the proportion of lactic acid units prevails and is at least 60%. The release rate from a PLGA polymer having 50% lactic acid units and 50% glycolic acid unit, under physiological conditions, is considerably shorter than for PLGA polymers having at least 60% lactic acid units, and therefore does not allow the active ingredient to be provided in sufficient quantities over an extended service life of the implant.

The PLGA polymer, polylactide-co-glycolide polymer, or also poly(lactic-co-glycolic acid) polymer, is a copolymer comprising lactic acid units and glycolic acid units. The PLGA polymer is a linear aliphatic polyester of the two monomer units, lactic acid and glycolic acids. The PLGA polymers can be characterized and differentiated based on the ratio of the two monomer units to each other. For example, a PLGA 50:50 polymer comprises 50% lactic acid units and 50% glycolic acid units, while a PLGA 85:15 polymer comprises 85% lactic acid units and 15% glycolic acid units. The PLGA polymer is biodegradable and can be hydrolyzed in the body into the original monomers, lactic acid and glycolic acid. PLGA polymers and/or the decomposition products thereof are characterized by very low toxicity.

According to the invention, PLGA polymers are used which have a ratio of the monomer units to each other of 60-99% lactic acid units to 40-1% glycolic acid units, preferably of 75-90% lactic acid units to 25-10% glycolic acid units, with 85% lactic acid units to 15% glycolic acid units being particularly preferred. They can allow release rates as long as magnesium degradation occurs.

The coating and/or cavity filling according to the invention comprise PLGA polymers and taxane embedded therein. This can be a certain taxane or a mixture of different taxanes. In addition to the taxane, other substances or compounds may be embedded in the PLGA polymer. Preferably substantially only taxane, and optionally adjuvants, are embedded in the selected PLGA polymer, such as preservatives or substances to stabilize the embedded taxane. For the purpose of the present invention, the term “taxane” shall denote chemical compounds that have a diterpene-containing skeleton and exhibit a cytotoxic or cytostatic activity. Preferred taxanes are those used for cancer therapy and comprise paclitaxel, docetaxel, larotaxel, ortataxel and/or tesetaxel, and the salts and/or derivatives thereof. In a particularly preferred embodiment, the taxane is paclitaxel and/or salts thereof.

The implant according to the invention has a certain release rate of the taxane or taxanes. After day two after the implantation, the release rate is ≦400 ng taxane per day for at least a period of more than 10 consecutive days. “After day two after the implantation” shall be interpreted as an in vivo release rate which occurs immediately or at a later time after the expiration of a 48-hour time period after implantation and can be measured or determined. The release rate according to the invention can be determined in vitro, such as plasma from pigs or humans.

According to the invention, the release rate is ≦400 ng taxane per day, preferably 50 ng-350 ng taxane per day, with 100-300 ng taxane per day being particularly preferred, in each case after day two after the implantation. This release rate can be observed for a time period of more than 10 consecutive days, and this release rate is preferably achieved over a period of more than 30 consecutive days, with a period of 30 to 120 consecutive days being particularly preferred. The corresponding time frame does not have to be immediately after the implantation or start on day 3 after the implantation, but may appear later in the elution profile.

The taxane can in particular be dispensed in a substantially linear manner over the defined time frame. The term “in a substantially linear manner” shall mean a release that can be determined by a graphical representation of the cumulatively measured released taxane quantities over the time, wherein the compensation degree, determined according to the least square method, for data points within the defined time frame has a correlation coefficient of greater than 0.9, preferably greater than 0.92, with greater than 0.95 being particularly preferred.

The implant is preferably made entirely or partially of a biocorrodible metallic material. This biocorrodible material is preferably a magnesium alloy.

The implant according to the invention is preferably a stent.

The implant preferably has a metallic base body. The metallic base body is in particular made of magnesium, a biocorrodible magnesium alloy, technical pure iron, a biocorrodible iron alloy, a biocorrodible tungsten alloy, a biocorrodible zinc alloy, or a biocorrodible molybdenum alloy.

Biocorrodible as defined by the invention denotes alloys and elements in the physiological environment of which resorption/reorganization takes place, so that the part of the implant made of the material is no longer present in its entirety, or at least predominantly.

In the present invention, a magnesium alloy, iron alloy, zinc alloy, molybdenum alloy, or tungsten alloy denotes a metallic structure comprising magnesium, iron, zinc, molybdenum or tungsten as the main constituent. The main constituent is the alloying constituent, the weight proportion of which is the highest in the alloy. A fraction of the main constituent is preferably more than 50 weight %, particularly more than 70 weight %. The alloy is to be selected in the composition thereof such that it is biocorrodible. A possible test medium for testing the corrosion behavior of a potential alloy is synthetic plasma, as that which is required according to EN ISO 10993-15:2000 for biocorrosion analyses (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). For this purpose, a sample of the alloy to be analyzed is stored in a closed sample container with a defined quantity of the test medium at 37° C. The samples are removed at intervals—which are adapted to the anticipated corrosion behavior—ranging from a few hours to several months and analyzed for traces of corrosion in the known manner. The synthetic plasma according to EN ISO 10993-15:2000 corresponds to a blood-like medium and thereby is a possible medium to reproducibly simulate a physiological environment as defined by the invention.

According to the invention, the implant has a coating or a cavity filling comprising or consisting of a PLGA polymer, in which a taxane is embedded. A coating as defined by the invention denotes an application of the constituents of the coating onto at least some regions of the base body of the implant. Preferably, the entire surface of the base body of the implant is covered by the coating. A layer thickness preferably ranges from 1 nm to 100 μm, with 300 nm to 15 μm being particularly preferred. The coating can be directly applied to the implant surface. The processing can be carried out according to standard coating methods. It is possible to produce single-layer or multi-layer system (such as so-called base coat, drug coat, or top coat layers). The coating can be directly applied onto the base body of the implant, or additional layers may be provided in between.

As an alternative or in addition, the taxane can be part of a cavity filling. The cavity is generally located on the surface of the implant. In the case of stents having a biodegradable base body, the cavity may also be disposed on the inside of the base body, so that the taxane is not released until the cavity is exposed. The cavity can be selectively open or covered by another coating.

Method for coating implants and for applying cavity fillings onto implants are known to the person skilled in the art.

In addition to using the taxanes according to the invention, the coating or cavity filling may comprise other constituents, and the coating or cavity filling can in particular comprise additional polymers, such as a polymeric, preferably organic carrier matrix. The coating and/or cavity filling can in particular also comprise additional pharmaceutical active ingredients, X-ray markers, or magnetic resonance markers.

### DESCRIPTION OF FIGURES

FIG. 1: Elution profile of an AMS coated with PLGA 50:50 and 10% paclitaxel, or PLGA 85:15 and 10% paclitaxel, wherein the release is stated cumulatively in % over time in hours (h).

FIG. 2: Elution profile of an AMS coated with PLGA 50:50 and 5% paclitaxel, or PLGA 85:15 and 7.5% paclitaxel, wherein the release is stated cumulatively in % over time in hours (h).

FIG. 3: Elution profile of an AMS coated with PLGA 50:50 and 5% paclitaxel, or PLGA 85:15 and 7.5% paclitaxel, wherein the release is stated cumulatively in % over time in hours (h).

### DETAILED DESCRIPTION

The invention will be explained in more detail hereinafter based on exemplary embodiments.

### Example 1

### Coating an AMS with PLGA 50:50 and 10% Paclitaxel

An AMS (3.0-10 mm) is coated with a solution of PLGA 50:50 in acetone comprising 10% paclitaxel relative to the polymer weight, up to a weight of the coating of 2.7 μg/mm². Afterwards, the coated stent is dried at 40° C. under vacuum for 13 hours.

### Example 2

### Coating an AMS with PLGA 85:15 and 10% Paclitaxel

An AMS (3.0-10 mm) is coated with a solution of PLGA 85:15 in acetone comprising 10% paclitaxel relative to the polymer weight, up to a weight of the coating of 2.7 μg/mm². Afterwards, the coated stent is dried at 40° C. under vacuum for 13 hours.

### Example 3

### Elution Profile of Coated AMS in Porcine Plasma

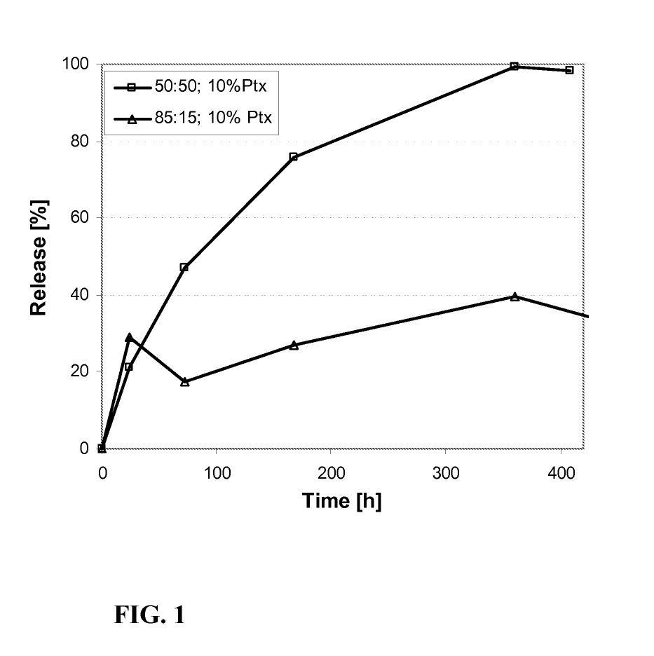

The elution of paclitaxel in porcine plasma was determined for AMS according to Example 1 and Example 2. The results are summarized in FIG. 1. It was shown that Paclitaxel initially elutes very quickly from PLGA 50:50, so that on day 3 as much as 50% of the paclitaxel quantity had been released from the coating, and as much as approximately 80% on day 7. After 15 days, substantially the entire paclitaxel content has been released from the coating. For PLGA 85:15 a comparatively slower release having a linear release curve was observed, following an initial increased release in the first two days, so that after 15 days only approximately 40% of the paclitaxel quantity was eluted from the coating.

### Example 4

### Evaluation of the Effect in the Pig Artery Model

Stents coated according to Example 1 or 2 were implanted into coronary arteries of Yucatan Mini Swine. For control purposes, an uncoated AMS was implanted. The oversize of the stents was 1.2 in each case. After one month, coronary angiography was conducted in the pigs. The pigs were subsequently killed and the coronary arteries were isolated and underwent a morphometric analysis. The results are summarized in Table 1.

TABLE 1

| | QCA Stenosis | Histomorphometry |
|---|---|---|
| Group | Diameter (%) | IEL Surface (mm²) |
| AMS control | 20 | 4.92 |
| PLGA 50:50; 10% Paclitaxel | 22 | 7.52* |
| PLGA 85:15; 10% Paclitaxel | 3* | 8.47* |

*p < 0.05 compared to the AMS control

### Example 5

### Coating an AMS with PLGA 50:50 and 5% Paclitaxel

An AMS (3.0-15 mm) is coated with a solution of PLGA 50:50 in acetone comprising 5% paclitaxel relative to the polymer weight, up to a weight of the coating of 3.3 μg/mm², Afterwards, the coated stent is dried at 40° C. under vacuum for 13 hours.

### Example 6

### Coating an AMS with PLGA 85:15 and 7.5% Paclitaxel

An AMS (3.0-15 mm) is coated with a solution of PLGA 85:15 in acetone comprising 7.5% paclitaxel relative to the polymer weight, up to a weight of the coating of 2.2 μg/mm². Afterwards, the coated stent is dried at 40° C. under vacuum for 13 hours.

### Example 7

### Elution Profile of Coated AMS in Porcine Plasma

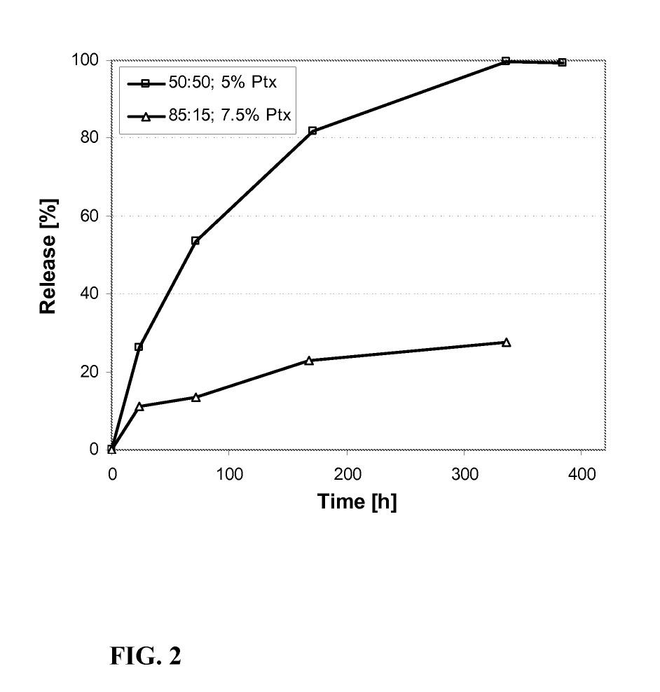

The elution of paclitaxel in porcine plasma was determined for AMS according to Example 5 and Example 6. The results are summarized in FIG. 2. It was shown that Paclitaxel initially elutes very quickly from PLGA 50:50, so that on day 3 as much as 50% of the paclitaxel quantity was released from the coating, and as much as approximately 80% on day 7. After 15 days, substantially the entire paclitaxel content had been released from the coating. For PLGA 85:15 a comparatively slower release having a linear release curve was observed, following an initial increased release in the first two days, so that after 15 days only approximately 30% of the paclitaxel quantity was eluted from the coating.

### Example 8

### Evaluation of the Effect in the Pig Artery Model

Stents coated according to Example 5 or 6 were implanted into coronary arteries of Yucatan Mini Swine. For control purposes, an uncoated AMS was implanted. The oversize of the stents was 1.2 in each case. After one month, coronary angiography was conducted in the pigs. The pigs were subsequently killed and the coronary arteries were isolated and underwent a morphometric analysis. The results are summarized in Table 2.

TABLE 2

| | QCA Stenosis | Histomorphometry |
|---|---|---|
| Group | Diameter (%) | Stent Surface (mm²) |
| AMS control | 21 | 4.32 |
| PLGA 50:50; 5.0% Paclitaxel | 45* | 4.70 |
| PLGA 85:15; 7.5% Paclitaxel | 5* | 5.67* |

*p < 0.05 compared to the AMS control

### Example 9

### Coating an AMS with PLGA 50:50 and 5% Paclitaxel

An AMS (3.0-16 mm) is coated with a solution of PLGA 50:50 in acetone comprising 5% paclitaxel relative to the polymer weight, up to a weight of the coating of 2.7 μg/mm². Afterwards, the coated stent is dried at 40° C. under vacuum for 13 hours.

### Example 10

### Coating an AMS with PLGA 85:15 and 7.5% Paclitaxel

An AMS (3.0-16 mm) is coated with a solution of PLGA 85:15 in acetone comprising 7.5% paclitaxel relative to the polymer weight, up to a weight of the coating of 1.0 μg/mm². Afterwards, the coated stent is dried at 40° C. under vacuum for 13 hours.

### Example 11

### Elution Profile of Coated AMS in Porcine Plasma

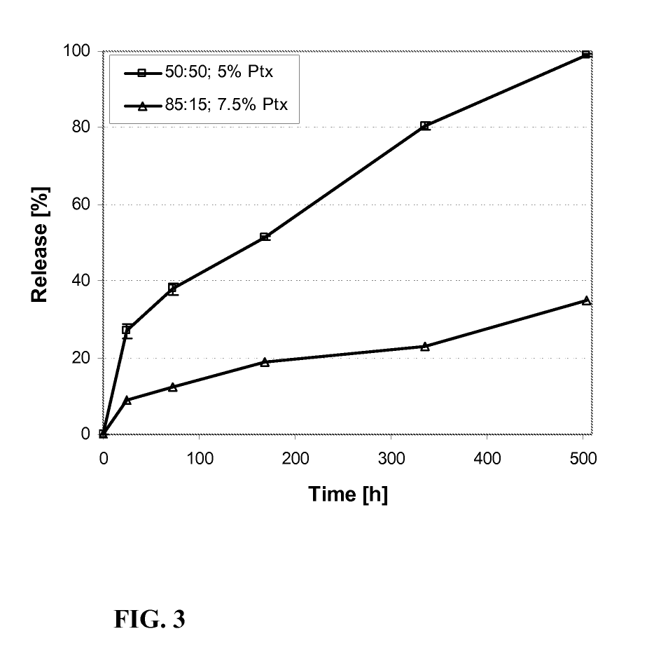

The elution of paclitaxel in porcine plasma was determined for AMS according to Example 9 and Example 10. The results are summarized in FIG. 3. It was shown that Paclitaxel initially elutes very quickly from PLGA 50:50, so that on day 3 as much as 40% of the paclitaxel quantity was released from the coating, and as much as approximately 50% on day 7. After 21 days, substantially the entire paclitaxel content had been released from the coating. For PLGA 85:15 a comparatively slower release having a linear release curve was observed, following an initial increased release in the first two days, so that after 21 days only approximately 30% of the paclitaxel quantity was eluted from the coating.

### Example 12

### Evaluation of the Effect in the Pig Artery Model

Stents coated according to Example 9 or 10 were implanted into coronary arteries of Yucatan Mini Swine. For control purposes, an uncoated AMS was implanted. The oversize of the stents was 1.2 in each case. After one and three months, coronary angiography was conducted in the pigs. The pigs were subsequently killed and the coronary arteries were isolated and underwent a morphometric analysis. The results are summarized in Table 2.

TABLE 2

| | QCA Stenosis | | Histomorphometry | |
|---|---|---|---|---|
| | Diameter [%] | | Stent Surface [mm²] | |
| Group | 28 d | 90 d | 28 d | 90 d |
| PLGA 50:50; 5.0% Paclitaxel | 3 | 32 | 6.7 | 4.6 |
| PLGA 85:15; 7.5% Paclitaxel | 3 | 25 | 6.6 | 6.0* |

*p < 0.05 compared to the 50:50; 5% Ptx

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An implant having a coating or a cavity filling comprising a PLGA polymer and taxane embedded therein, the release rate of the taxane after day two after implantation being 400 ng/day for a period or more than 10 consecutive days, characterized in that the PLGA polymer has a ratio of the monomer units among each other of 60-99% lactic acid units to 40-1% glycolic acid units.

2. The implant according to claim 1, characterized in that the implant is made entirely or partially of a biocorrodible metallic material.

3. An implant according to claim 1, characterized in that the biocorrodible metallic material is a magnesium alloy.

4. An implant according to claim 1, characterized in that the implant is a stent.

5. An implant according to claim 1, characterized in that the taxane is paclitaxel or a salt thereof.

6. An implant according to claim 1, characterized in that the PLGA polymer of the coating or cavity filling of the implant has a ratio of the monomer units to each other of 75-90% lactic acid units to 25-10 glycolic acid units.

7. An implant according to claim 1, characterized in that the PLGA polymer of the coating or cavity filling of the implant has a ratio of the monomer units to each other of 85% lactic acid units to 15% glycolic acid units.

8. An implant according to claim 1, characterized in that the taxane is released after day two after implantation at a rate of 50 ng-350 ng/day, optionally 100-300 ng/day.

9. An implant according to claim 1, characterized in that the release rate is reached over a period of more than 30 consecutive days.

10. The implant according to claim 9, characterized in that the release rate is reached over a period of 30 to 120 consecutive days.

11. An implant according to claim 1, characterized in that after day two after implantation the taxane is dispensed in a substantially linear fashion for the defined period.
